(19) United States
(12) Patent Application Publication
(10) Pub. No.: US 2024/0067604 A1
(43) Pub. Date: Feb. 29, 2024

(54) COMBINATIONS OF IRS/STAT3 DUAL MODULATORS AND ANTI PD-1/PD-L1 ANTIBODIES FOR TREATING CANCER

(71) Applicant: TYRNOVO LTD., Tel Aviv (IL)

(72) Inventors: Hadas REUVENI, Har Adar (IL); Lana Kupershmidt, Kiryat Biyalik (IL); Izhak Haviv, Rosh Pine (IL)

(73) Assignee: TYRNOVO LTD., Tel Aviv (IL)

(21) Appl. No.: 18/492,339

(22) Filed: Oct. 23, 2023

Related U.S. Application Data
(63) Continuation of application No. 16/764,246, filed on May 14, 2020.

(51) Int. Cl. C07C 327/44; A61P 35/00; C07D 279/08; C07K 16/28

(52) U.S. Cl. CPC C07C 327/44; A61P 35/00; C07D 279/08; C07K 16/2818

(57) ABSTRACT

The present invention relates to the treatment of cancer using combination therapy comprising a dual modulator of Insulin Receptor Substrate (IRS) and signal transducer and activator of transcription 3 (Stat3), in combination with an antibody against programmed cell death 1 (PD-1) protein, an anti-programmed cell death protein 1 ligand (PD-L1) antibody, or a combination thereof. The combination can be used to re-sensitize a tumor that may develop or has developed resistance to the anti-PD-1 and/or anti-PD-L1 antibody, by enhancing response of the tumor to the anti-PD-1 and/or anti-PD-L1 antibody, converting non-responding tumors to responders and/or blocking tumor progression.

### CROSS REFERENCE TO RELATED APPLICATIONS

This application is a Continuation of U.S. application Ser. No. 16/764,246, filed on May 14, 2020; which is a national phase of PCT International Application No. PCT/IL2017/051249, filed on Nov. 16, 2017, which are hereby incorporated by reference in their entirety.

### FIELD OF THE INVENTION

The present invention relates to the treatment of cancer using combination therapy comprising a dual modulator of Insulin Receptor Substrate (IRS) and signal transducer and activator of transcription 3 (Stat3), in combination with an antibody against programmed cell death 1 protein (PD-1) and/or an antibody against programmed cell death protein 1 ligand (PD-L1). The combination can be used to re-sensitize a tumor that may develop or has developed resistance to the anti-PD-1 and/or anti-PD-L1 antibody, by enhancing response of the tumor to the anti-PD-1 antibody, converting non-responding tumors to responders and/or blocking tumor progression.

### BACKGROUND OF THE INVENTION

Tyrphostins are a family of protein tyrosine kinase inhibitors, designed to mimic the tyrosine substrate, the ATP and can inhibit allosterically the enzyme (Levitzki et al., Science (1995), 267:1782-88; Levitzki et al., Biochem. Pharm. (1990), 40:913-920; Levitzki et al., FASEB J. (1992), 6:3275-3282; U.S. Pat. Nos. 5,217,999 and 5,773,476, Posner et al., Mol. Pharmacol. (1994), 45:673-683). The pharmacophores of these tyrphostins, and in particular tyrphostins of the benzylidene malonitril type, are the hydrophilic catechol ring and the more lipophilic substituted cyano-vinyl radical. Kinetic studies have shown that some tyrphostin compounds are pure competitive inhibitors vis-à-vis tyrosine substrates whereas for the ATP binding site they act as non-competitive inhibitors (Yaish et al., Science (1988), 242:933-935; Gazit et al., J. Med. Chem. (1989), 32:2344-2352). Nonetheless, many tyrphostins have shown competitive inhibition against both the substrate and ATP binding site or mixed competitive (Posner et al., Mol. Pharmacol. (1994), 45:673-683).

In a related group of tyrphostins, the hydrophilic catechol ring was exchanged by lipophilic dichloro- or dimethoxy-phenyl groups, to yield EGFR kinase inhibitors, effective in the low micromolar range (Yoneda et al., Cancer Res. (1991), 51: 4430-4435). These tyrphostins were further administered to tumor-bearing nude mice together with anti-EGFR monoclonal antibodies at a suboptimal dose to afford markedly enhanced inhibition of tumor growth.

WO 2008/068751 to some of the inventors of the present invention, discloses compounds having increased inhibitory properties of insulin-like growth factor 1 receptor (IGF1R), platelet derived growth factor receptor (PDGFR), epidermal growth factor receptor (EGFR), and IGF1R-related insulin receptor (IR) activation and signaling.

WO 2009/147682 to some of the inventors of the present invention discloses compounds acting as protein kinase (PK) and receptor kinase (RK) signaling modulators. Further disclosed in WO 2009/147682 are methods of preparation of the such compounds, pharmaceutical compositions including such compounds, and methods of using these compounds and compositions, especially as chemotherapeutic agents for preventions and treatments of PK and RK related disorders such as metabolic, inflammatory, fibrotic, and cell proliferative disorders, in particular cancer.

WO 2012/117396 to some of the inventors of the present invention describes combinations of the compounds of WO 2008/068751 or WO 2009/147682 with anti-cancer agents for the treatment of cancer.

WO 2016/125169 to some of the inventors of the present invention describes combinations of the compounds of WO 2008/068751 or WO 2009/147682 with (i) an Epidermal Growth Factor inhibitor (EGFR inhibitor) and EGFR antibody; (ii) an inhibitor of mammalian target of rapamycin (mTOR); (iii) a mitogen-activated protein kinase (MEK) inhibitor; (iv) a mutated B-Raf inhibitor; (v) an immunotherapy agent; and (vi) a chemotherapeutic agent for the treatment of cancer.

In the last few decades immunotherapy has become an important part of treating some types of cancer. The goal of cancer immunotherapy is to enable the patient's immune system to specifically recognize and kill cancer cells. Signal transducer and activator of transcription 3 (Stat3) is often activated in cancer and directly involved in the implementation and maintenance of the cancer immunosuppressive microenvironment and plays a central role in tumor immune evasion.

There is an unmet need for combinations that are useful for treating cancer, preferably providing at least additive therapeutic effects. Combinations of drugs from different categories are useful to prevent or overcome emergence of drug resistant tumors.

### SUMMARY OF THE INVENTION

The present invention relates to the treatment of cancer using combination therapy comprising a dual modulator of Insulin Receptor Substrate (IRS) and signal transducer and activator of transcription 3 (Stat3), in combination with an antibody against programmed cell death 1 (PD-1) protein. The combination can be used to re-sensitize a tumor that may develop or has developed resistance to the anti-PD-1 and/or anti-PD-L1 antibody, by enhancing response of the tumor to the anti-PD-1 and/or anti-PD-L1 antibody, converting non-responding tumors to responders and/or blocking tumor progression.

The compounds described herein are modulators of Insulin Receptor Substrate 1 (IRS1) and/or Insulin Receptor Substrate 2 (IRS2) signaling. Accordingly, these compounds are referred to herein as “modulators of IRS”. In some embodiments, the compounds are inhibitors of IRS1 and/or IRS2. In further embodiments, the compounds of the invention are inhibitors of insulin-like growth factor 1 receptor (IGF-1R). As such, these compounds are useful in inhibiting, treating or preventing IGF-1R and/or IRS1 and/or IRS2 signaling related disorders, for example cancer. In some embodiments, the compounds trigger any one or more of the following, in any order: (i) dissociation of IRS1 and/or IRS2 from the cell membrane; (ii) serine phosphorylation of the IGF-1R direct substrates IRS1 and/or IRS2; and/or (iii) degradation of IRS1 and/or IRS2, thus providing long-lasting effects which enhance the inhibitory activity of these compounds. In other embodiments, the compounds are also inhibitors of IGF1R-related insulin receptor (IR), or proteins affected by or mediated by these PTKs or that are part of the PTK-mediated signal transduction pathway.

The compounds described herein are also modulators of signal transducer and activator of transcription 3 (Stat3). Accordingly, these compounds are also referred to herein as “modulators of Stat3”. In some embodiments, the compounds lead to the inhibition of Stat3 phosphorylation in cancer cells. Increased levels of Stat3 phosphorylation are detected in various cancers and drug-resistant cancers, leading to enhanced cancer survival. Moreover, treatment of cancers with PK inhibitor drugs surprisingly leads to the induction of Stat3 phosphorylation, as demonstrated herein. Without wishing to be bound by any particular theory or mechanism of action, it is contemplated that inhibiting Stat3 activity with the compounds of the present invention may synergize with such PK inhibitor drugs, which as a side effect upregulate Stat3, may prevent acquired resistance to such drugs, and may be effective for drug-resistant cancers.

Due to their dual effect on IRS and Stat3, the compounds are further described herein as “IRS/Stat3 dual modulators”.

It has been found that dual modulators of IRS and Stat3 can be used to sensitize a tumor to immunotherapy against anti-PD-1. Stat3 is often activated in cancer and is directly involved in the implementation and maintenance of the cancer immunosuppressive microenvironment and plays a central role in tumor immune evasion. Without wishing to be bound by any particular theory or mechanism of action, it is contemplated that inhibition of Stat3 phosphorylation with the compounds of the present invention un-masks the tumor from the local immune system and sensitizes them to immunotherapy to antibodies against PD-1.

In accordance with the principles of the present invention, it has now been demonstrated that a compound of formula 4, which is a representative of compounds of formula (I) as described herein, in combination with Pembrolizumab (Keytruda®), a monoclonal antibody against PD-1, converted non-responding tumors to responders and blocked tumor progression in a patient-derived xenograft (PDX) model of immune-deficient mice, in which a tumor originated from an esophagus cancer biopsy was implanted, and peripheral blood mononuclear cells (PBMCs) isolated from the blood of the same patients were injected to the mice on the first day of treatment. While no response was observed with Keytruda® alone or with Compound 4 alone, and the tumors aggressively progressed, mice treated concomitantly with a combination of Keytruda® and Compound 4 demonstrated complete blockage of tumor progression. This demonstrates the ability of the compound to overcome cancer drug resistance to anti-PD-1 antibody therapy.

Thus, in one embodiment, the present invention relates to a pharmaceutical combination comprising a compound represented by the structure of formula (I), or salts or hydrates thereof, in combination with an anti-programmed cell death protein 1 (PD-1) antibody, an anti-programmed cell death protein 1 ligand (PD-L1) antibody or a combination thereof

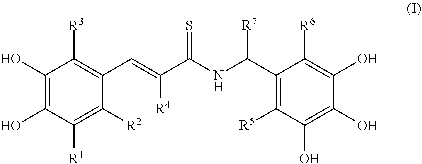

wherein
R¹, R², R³, R⁵ and R⁶ are each independently selected from the group consisting of H, halogen, haloalkyl and OR¹⁶ wherein R¹⁶ is H or C₁-C₄ alkyl;
R⁴ is H or CN; and
R⁷ is H or C₁-C₄ alkyl.

In some embodiments, the compound is represented by the structure of any of formulae 1, 2, 3, 4, 5, 6, 7, 8, 9, 10, 11, 12, 13, 14, 15 or 16. The structures of these compounds are set forth in the detailed description hereinbelow. A currently preferred combination comprises a compound of formula 4.

In another embodiment, the present invention relates to a pharmaceutical combination comprising a compound represented by the structure of formula (I), or salts or hydrates thereof, in combination with an anti-programmed cell death protein 1 (PD-1) antibody, an anti-programmed cell death protein 1 ligand (PD-L1) antibody or a combination thereof

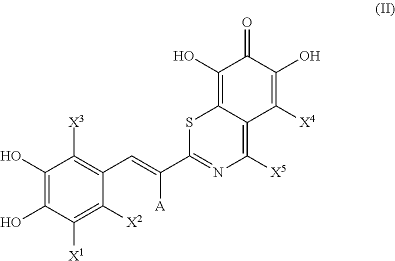

wherein
A is H or CN;
X¹, X², X³ and X⁴ are each independently selected from H, halogen, C₁-C₄ alkyl, haloalkyl and OR¹ wherein R¹ is H or C₁-C₄ alkyl; and
X⁵ is H or C₁-C₄ alkyl.

In some embodiments, the compound is represented by the structure of any of formulae 17, 18, 19, 20, 21, 22, 23, 24 or 25. The structures of these compounds are set forth in the detailed description hereinbelow. A currently preferred combination comprises a compound of formula 20.

In another embodiment, the present invention further relates to method of sensitizing a tumor to immunotherapy by an anti-programmed cell death protein 1 (PD-1) antibody, an anti-programmed cell death protein 1 ligand (PD-L1) antibody or a combination thereof, the method comprising the step of contacting the tumor with a compound represented by the structure of formula (I) or (II), or salts or hydrates thereof, in combination with an anti-programmed cell death protein 1 (PD-1) antibody and/or an anti-programmed cell death protein 1 ligand (PD-L1) antibody.

In other embodiments, the present invention further relates to a combination comprising a compound of formula (I) or (II) with an anti-programmed cell death protein 1 (PD-1) antibody, an anti-programmed cell death protein 1 ligand (PD-L1) antibody or a combination thereof, for use in sensitizing a tumor to immunotherapy with the anti-PD-1 antibody.

A currently preferred compound for use in the combinations of the present invention is a compound of formula 4. Thus, in another embodiment, the present invention relates to a pharmaceutical combination comprising a compound represented by the structure of formula 4, or salts or hydrates thereof, in combination with an anti-programmed cell death protein 1 (PD-1) and/or anti-programmed cell death protein 1 ligand (PD-L1) antibody.

In another embodiment, the present invention further relates to method of sensitizing a tumor to immunotherapy by an anti-programmed cell death protein 1 (PD-1) antibody, an anti-programmed cell death protein 1 ligand (PD-L1) antibody or a combination thereof, the method comprising the step of contacting the tumor with a compound represented by the structure of formula 4, or salts or hydrates thereof, in combination with an anti-programmed cell death protein 1 (PD-1) and/or anti-programmed cell death protein 1 ligand (PD-L1) antibody.

In other embodiments, the present invention further relates to a combination comprising a compound of formula 4 with an anti-programmed cell death protein 1 (PD-1) antibody, an anti-programmed cell death protein 1 ligand (PD-L1) antibody or a combination thereof, for use in sensitizing a tumor to immunotherapy with the anti-PD-1 and/or anti-PD-L1 antibody.

The structure of the compound of formula 4 is described hereinbelow:

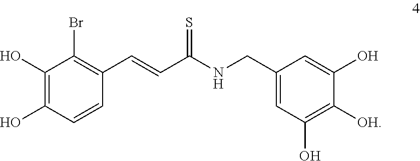 [SMILES: Oc1cc(CNC(=S)/C=C/c2ccc(O)c(O)c2Br)cc(O)c1O]

In some embodiments, the anti-PD-1 antibody used in combination with the compound described above is selected from the group consisting of selected from the group consisting of Pembrolizumab (Keytruda®), Nivolumab (Opdivo®), Pidilizumab (CT-011, MDV9300, Medivation), AGEN-2034, AMP-224, BCD-100, BGBA-317, BI-754091, CBT-501, CC-90006, Cemiplimab, GLS-010, IBI-308, JNJ-3283, JS-001, MEDI-0680, MGA-012, MGD-013, PDR-001, PF-06801591, REGN-2810, SHR-1210, TSR-042, LZM-009 and ABBV-181. Each possibility represents a separate embodiment of the present invention.

In a currently preferred embodiment, the anti-PD-1 antibody is Pembrolizumab (Keytruda®). Thus, a currently preferred combination comprises a compound of formula 4 and Pembrolizumab.

In some embodiments, the anti-PD-L1 antibody used in combination with the compound described above is selected from the group consisting of Durvalumab (Imfinzi™), Atezolizumab (Tecentriq®, MPDL3280A), Avelumab (Bavencio®), CX-072, BMS-936559, SHR-1316, M-7824, LY-3300054, FAZ-053, KN-035, CA-170, CK-301, CS-1001, HLX-10, MCLA-145, MSB-2311 and MEDI-4736. Each possibility represents a separate embodiment of the present invention.

In some embodiments, the tumor is present in a cancer patient who is receiving immunotherapy by an anti-PD-1 and/or anti-PD-L1 antibody, or a candidate for receiving such immunotherapy. In some embodiments, the tumors in the cancer patients are resistant to treatment with the anti-PD-1 and/or anti-PD-L1 antibody alone. As contemplated herein, the compound of formula (I) or (II), especially compound 4, re-sensitizes the tumor to immunotherapy by the anti-PD-1 and/or anti-PD-L1 antibody by enhancing response of the tumor to the anti-PD-1 and/or anti-PD-L1 antibody, converting non-responding tumors to responders and/or blocking tumor progression.

The combinations of the present invention are suitable for treating various types of cancers. In particular, the combinations of the present invention are active against head and neck (H&N) cancer, sarcoma, multiple myeloma, ovarian cancer, breast cancer, kidney cancer, stomach cancer, hematopoietic cancers, lymphoma, leukemia, including lymphoblastic leukemia, lung carcinoma, melanoma, glioblastoma, hepatocarcinoma, prostate cancer, pancreatic cancer and colon cancer. Each possibility represents a separate embodiment of the present invention.

The term “combination” or “combined treatment” as used herein denotes any form of concurrent or parallel treatment with at least two distinct therapeutic agents. This term is intended to encompass both concomitant administration of the two treatment modalities, i.e., using substantially the same treatment schedule, as well as overlapping administration in sequential or alternating schedules of each treatment. Each possibility represents a separate embodiment of the present invention.

The combination therapy is particularly advantageous, since the dosage of each agent in a combination therapy can be reduced as compared to mono-therapy with each agent, while still achieving an overall anti-cancer effect. Accordingly, reducing the dosage of each agent may result in decreased side effects. The combination therapy may reduce the development of resistance to a specific anti-cancer treatment and/or lead to regression of the tumor after it has acquired resistance, as demonstrated herein.

The compound of formula (I) or (II) (e.g., compound 4) and the anti-PD-1 and/or anti-PD-L1 antibody can be administered simultaneously (in the same or in separate dosage forms), or they can be administered sequentially, in any order. The administration can also take place according to alternating dosing schedules, e.g., compound of formula (I) or (II) followed by anti-PD-1 and/or anti-PD-L1 antibody, then an additional dose of the compound of formula (I) or (II), followed by the same or another anti-PD-1 and/or anti-PD-L1 antibody, and so forth. All administration schedules, including simultaneous, sequential and alternating, are contemplated by the present invention, wherein each possibility represents a separate embodiment of the present invention.

The pharmaceutical compositions of the present invention can be provided in any form known in the art, for example in a form suitable for oral administration (e.g., a solution, a suspension, a syrup, an emulsion, a dispersion, a tablet, a pill, a capsule, a pellet, granules and a powder), for parenteral administration (e.g., intravenous, intramuscular, intra-arterial, transdermal, subcutaneous or intra-peritoneal), for topical administration (e.g., an ointment, a gel, a cream), for administration by inhalation or for administration via suppository. Each possibility represents a separate embodiment of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 depicts compound 4 in combination with Pembrolizumab (Keytruda®) converted non-responding tumors to responders and blocked tumor progression in a patient-derived xenograft (PDX) model of immune-deficient mice, in which a tumor originated from an esophagus cancer biopsy was implanted. The mice were supplemented with immune cells from the same patient (double autologous) on the first day of treatment. Mice were treated with (a) Control (vehicle) (⋄); (b) Keytruda® (□); (c) Compound 4 (Δ); or (d) Keytruda®+Compound 4 (○). Treatments were initiated when average tumor size was ˜160 mm³. While no response was observed with Keytruda® alone or with Compound 4 alone, and the tumors aggressively progressed, mice treated concomitantly with a combination of Keytruda® and Compound 4 demonstrated complete blockage of tumor progression.

### DETAILED DESCRIPTION OF THE INVENTION

The present invention relates to the treatment of cancer using combination therapy comprising a dual modulator of Insulin Receptor Substrate (IRS) and signal transducer and activator of transcription 3 (Stat3), in combination with an antibody against programmed cell death 1 (PD-1) protein and/or an anti-programmed cell death protein 1 ligand (PD-L1) antibody. The combination can be used to re-sensitize a tumor that has developed resistance to the anti-PD-1 and/or anti-PD-L1 antibody, by enhancing response of the tumor to the anti-PD-1 and/or anti-PD-L1 antibody, converting non-responding tumors to responders and/or blocking tumor progression.

### PD-1, PD-L1, and Anti-PD-1 and PD-L1 Antibodies

Programmed cell death protein 1, also known as PD-1, is a cell surface receptor that plays an important role in down-regulating the immune system and promoting self-tolerance by suppressing T cell inflammatory activity. Binding of the PD-1 ligands, PD-L1 and PD-L2 to the PD-1 receptor found in T cells inhibits T-cell proliferation and cytokine production. Upregulation of PD-1 ligands occurs in some tumors and signaling through this pathway can contribute to inhibition of active T-cell immune surveillance of tumors. Anti-PD-1 antibodies bind to the PD-1 receptor and block its interaction with PD-L1 and PD-L3, releasing PD-1 pathway-mediated inhibition of the immune response, including the anti-tumor immune response.

In some embodiments, the anti-PD-1 antibody used in combination with the compound described above is Pembrolizumab (Keytruda®). In other embodiments, the anti-PD-1 antibody used in combination with the compound described above is Nivolumab (Opdivo®). In other embodiments, the anti-PD-1 antibody used in combination with the compound described above is Pidilizumab (Medivation).

Additional PD-1 antibodies are selected from the group consisting of AGEN-2034 (Agenus), AMP-224 (Medimmune), BCD-100 (Biocad), BGBA-317 (Beigene), BI-754091 (Boehringer Ingelheim), CBT-501 (Genor Biopharma), CC-90006 (Celgene), cemiplimab (Regeneron Pharmaceuticals), durvalumab+MEDI-0680 (Medimmune), GLS-010 (Harbin Gloria Pharmaceuticals), IBI-308 (Eli Lilly), JNJ-3283 (Johnson & Johnson), JS-001 (Shanghai Junshi Bioscience Co.), MEDI-0680 (Medimmune), MGA-012 (MacroGenics), MGD-013 (Marcogenics), pazopanib hydrochloride+pembrolizumab (Novartis), PDR-001 (Novartis), PF-06801591 (Pfizer), REGN-2810 (Regeneron), SHR-1210 (Jiangsu Hengrui Medicine Co.), TSR-042 (Tesaro Inc.), LZM-009 (Livzon Pharmaceutical Group Inc) and ABBV-181 (AbbVie Inc). Each possibility represents a separate embodiment of the present invention.

In a currently preferred embodiment, the anti-PD-1 antibody is Pembrolizumab (Keytruda®).

In other embodiments, the anti-PD-L1 antibody used in the combinations of the present invention is selected from the group consisting of Durvalumab (MedImmune LLC), Atezolizumab (Hoffmann-La Roche Ltd, Chugai Pharmaceutical Co Ltd), Avelumab (Merck KGaA), CX-072 (CytomX Therapeutics Inc), BMS-936559 (ViiV Healthcare Ltd), SHR-1316 (Jiangsu Hengrui Medicine Co Ltd), M-7824 (Merck KGaA), LY-3300054 (Eli Lilly and Co), FAZ-053 (Novartis AG), KN-035 (AlphaMab Co Ltd), CA-170 (Curis Inc), CK-301 (TG Therapeutics Inc), CS-1001 (CStone Pharmaceuticals Co Ltd), HLX-10 (Shanghai Henlius Biotech Co Ltd), MCLA-145 (Merus NV), MSB-2311 (MabSpace Biosciences (Suzhou) Co Ltd) and MEDI-4736 (Medimmune).

As contemplated herein, the tumor being treated by the combination of the invention is present in a cancer patient who is receiving immunotherapy by an anti-PD-1 and/or anti-PD-L1 antibody, or a candidate for receiving such immunotherapy. In some embodiments, the tumors in the cancer patients are resistant to treatment with the anti-PD-1 and/or anti-PD-L1 antibody alone. As demonstrated herein, the compound of formula (I) or (II), e.g., compound 4, re-sensitizes the tumor to immunotherapy by the anti-PD-1 and/or anti-PD-L1 antibody by enhancing response of the tumor to the anti-PD-1 and/or anti-PD-L1 antibody, converting non-responding tumors to responders and/or blocking tumor progression. Each possibility represents a separate embodiment of the present invention.

### Insulin Receptor Substrate (IRS)/Signal Transducer and Activator of Transcription 3 (Stat3) Dual Modulators

Any compound of the general structure of formula (I) or (II), or any individual compound encompassed by such formula can be used in the compositions and methods of the present invention:

The structure of formula (I) is represented hereinbelow:

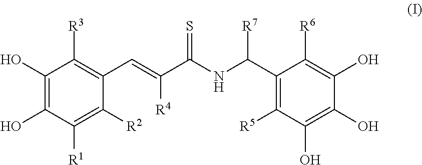

wherein
R¹, R², R³, R⁵ and R⁶ are each independently selected from the group consisting of H, halogen, haloalkyl and OR¹⁶ wherein R¹⁶ is H or C₁-C₄ alkyl;
R⁴ is H or CN; and
R⁷ is H or C₁-C₄ alkyl;
   and salts, hydrates and solvates thereof.

In other embodiments, the compound is a compound of formula (I) wherein at least one of R¹, R², R³, R⁵ and R⁶ is a halogen. The halogen may be F, Cl, Br or I, with each possibility representing a separate embodiment of the present invention.

In other embodiments, the compound is a compound of formula (I) wherein at least one of
R¹, R², R³, R⁵ and R⁶ is a haloalkyl.

In one embodiment, the compound is a compound of formula (I) wherein R¹ is H. In another embodiment, the compound is a compound of formula (I) wherein R¹ is halogen. In another embodiment, the compound is a compound of formula (I) wherein R¹ is a haloalkyl. In one particular embodiment, R¹ is F. In another particular embodiment, R¹ is Cl. In another particular embodiment, R¹ is Br. In another particular embodiment, R¹ is I. In another particular embodiment, R¹ is CF₃.

In another embodiment, the compound is a compound of formula (I) wherein R² is H. In another embodiment, the compound is a compound of formula (I) wherein R² is halogen. In one particular embodiment, R² is Br.

In another embodiment, the compound is a compound of formula (I) wherein R³ is H. In another embodiment, the compound is a compound of formula (I) wherein R³ is halogen. In one particular embodiment, R³ is Cl. In another particular embodiment, R³ is Br. In another particular embodiment, R³ is I.

In another embodiment, the compound is a compound of formula (I) wherein R⁴ is H. In another embodiment, the compound is a compound of formula (I) wherein R⁴ is CN.

In another embodiment, the compound is a compound of formula (I) wherein R⁵ is H.

In another embodiment, the compound is a compound of formula (I) wherein R⁶ is H. In another embodiment, the compound is a compound of formula (I) wherein R⁶ is halogen. In one particular embodiment, R⁶ is Br.

In another embodiment, the compound is a compound of formula (I) wherein R⁷ is H. In another embodiment, the compound is a compound of formula (I) wherein R⁷ is C₁-C₄ alkyl. In one particular embodiment, R⁷ is CH₃. In another particular embodiment, R⁷ is CH₂CH₃.

In one embodiment, the compound is a compound represented by formula 1:

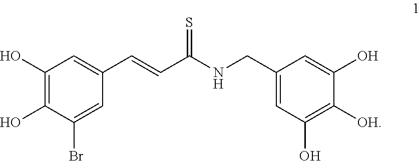 [SMILES: Oc1cc(CNC(=S)/C=C/c2cc(O)c(O)c(Br)c2)cc(O)c1O]

In another embodiment, the compound is a compound represented by formula 2:

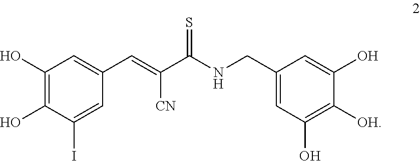 [SMILES: N#C/C(=C\c1cc(O)c(O)c(I)c1)C(=S)NCc1cc(O)c(O)c(O)c1]

In another embodiment, the compound is a compound represented by formula 3:

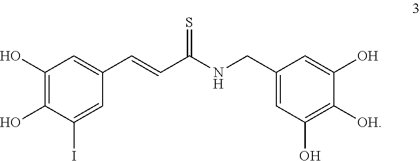 [SMILES: Oc1cc(CNC(=S)/C=C/c2cc(O)c(O)c(I)c2)cc(O)c1O]

In another embodiment, the compound is a compound represented by formula 4:

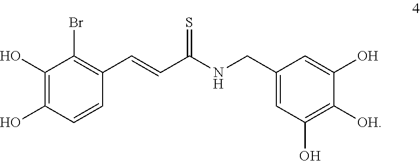 [SMILES: Oc1cc(CNC(=S)/C=C/c2ccc(O)c(O)c2Br)cc(O)c1O]

In another embodiment, the compound is a compound represented by formula 5:

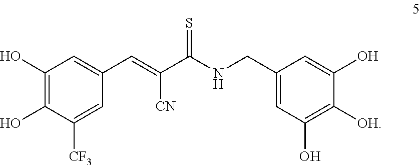 [SMILES: N#C/C(=C\c1cc(O)c(O)c(C(F)(F)F)c1)C(=S)NCc1cc(O)c(O)c(O)c1]

In another embodiment, the compound is a compound represented by formula 6:

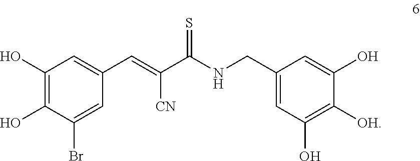 [SMILES: N#C/C(=C\c1cc(O)c(O)c(Br)c1)C(=S)NCc1cc(O)c(O)c(O)c1]

In another embodiment, the compound is a compound represented by formula 7:

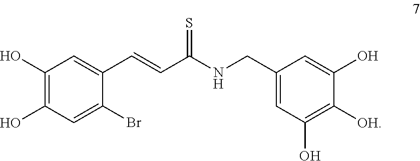 [SMILES: Oc1cc(Br)c(/C=C/C(=S)NCc2cc(O)c(O)c(O)c2)cc1O]

In another embodiment, the compound is a compound represented by formula 8:

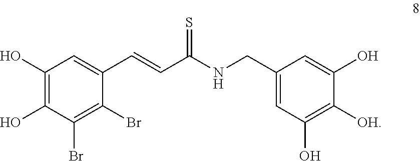 [SMILES: Oc1cc(CNC(=S)/C=C/c2cc(O)c(O)c(Br)c2Br)cc(O)c1O]

In another embodiment, the compound is a compound represented by formula 9:

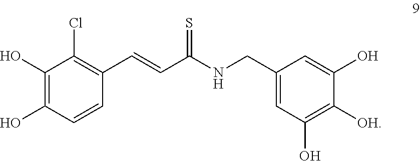 [SMILES: Oc1cc(CNC(=S)/C=C/c2ccc(O)c(O)c2Cl)cc(O)c1O]

In another embodiment, the compound is a compound represented by formula 10:

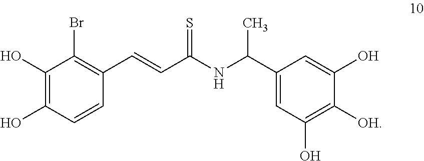 [SMILES: CC(NC(=S)/C=C/c1ccc(O)c(O)c1Br)c1cc(O)c(O)c(O)c1]

In another embodiment, the compound is a compound represented by formula 11:

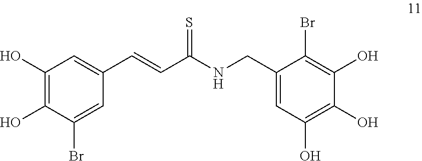 [SMILES: Oc1cc(/C=C/C(=S)NCc2cc(O)c(O)c(O)c2Br)cc(Br)c1O]

In another embodiment, the compound is a compound represented by formula 12:

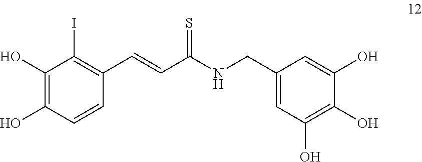 [SMILES: Oc1cc(CNC(=S)/C=C/c2ccc(O)c(O)c2I)cc(O)c1O]

In another embodiment, the compound is a compound represented by formula 13:

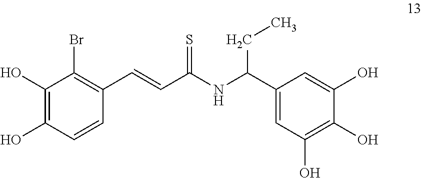 [SMILES: CCC(NC(=S)/C=C/c1ccc(O)c(O)c1Br)c1cc(O)c(O)c(O)c1]

In another embodiment, the compound is a compound represented by formula 14:

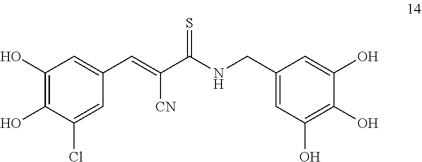 [SMILES: N#C/C(=C\c1cc(O)c(O)c(Cl)c1)C(=S)NCc1cc(O)c(O)c(O)c1]

In another embodiment, the compound is a compound represented by formula 15:

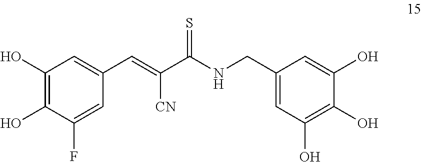 [SMILES: N#C/C(=C\c1cc(O)c(O)c(F)c1)C(=S)NCc1cc(O)c(O)c(O)c1]

In another embodiment, the compound is a compound represented by formula 16:

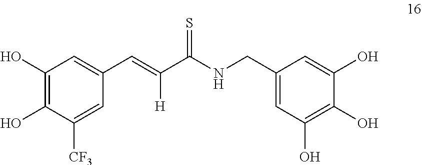 [SMILES: Oc1cc(CNC(=S)/C=C/c2cc(O)c(O)c(C(F)(F)F)c2)cc(O)c1O]

Each possibility represents a separate embodiment of the present invention.

In another embodiment, the compound is represented by the structure of formula (II):

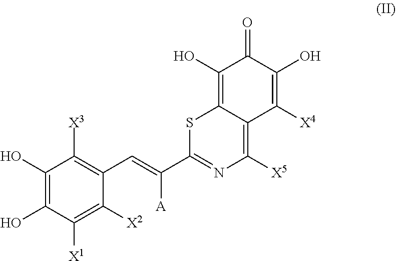

wherein
A is H or CN;
X¹, X², X³ and X⁴ are each independently selected from H, halogen, C₁-C₄ alkyl, haloalkyl and OR¹ wherein R¹ is H or C₁-C₄ alkyl; and
X⁵ is H or C₁-C₄ alkyl;
including salts, hydrates, solvates, polymorphs, optical isomers, geometrical isomers, enantiomers, diastereomers, and mixtures thereof.

In some embodiments, the compound is a compound of formula (II) wherein A is H.

In other embodiments, the compound is a compound of formula (II) wherein A is CN.

In other embodiments, the compound is a compound of formula (II) wherein at least one of X¹, X², X³ and X⁴ is a halogen. The halogen may be F, Cl, Br or I, with each possibility representing a separate embodiment of the present invention.

In other embodiments, the compound is a compound of formula (II) wherein X¹, X², X³ and X⁴ are each H or a halogen, wherein the halogen is preferably Cl, Br or I.

In other embodiments, the compound is a compound of formula (II) wherein X² is H.

In other embodiments, the compound is a compound of formula (II) wherein X⁵ is H.

In other embodiments, the compound is a compound of formula (II) wherein X⁵ is C₁-C₄.

Each possibility represents a separate embodiment of the present invention.

In one embodiment, the compound is a compound represented by formula 17:

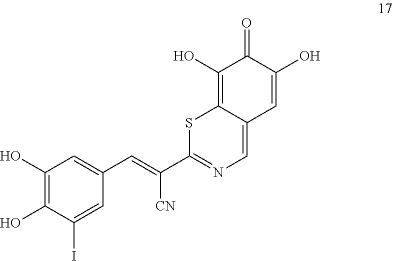 [SMILES: N#C/C(=C\c1cc(O)c(O)c(I)c1)c1ncc2cc(O)c(=O)c(O)c-2s1]

In another embodiment, the compound is a compound represented by formula 18:

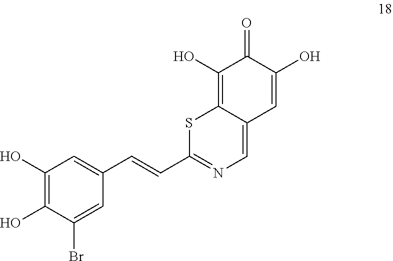 [SMILES: O=c1c(O)cc2cnc(/C=C/c3cc(O)c(O)c(Br)c3)sc-2c1O]

In another embodiment, the compound is a compound represented by formula 19:

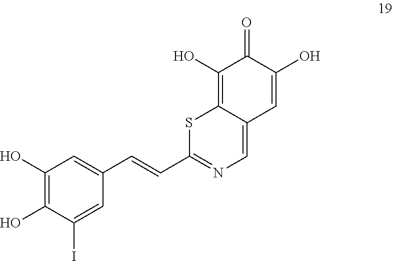 [SMILES: O=c1c(O)cc2cnc(/C=C/c3cc(O)c(O)c(I)c3)sc-2c1O]

In another embodiment, the compound is a compound represented by formula 20:

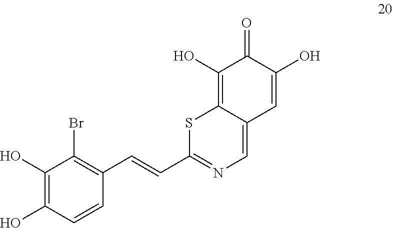 [SMILES: O=c1c(O)cc2cnc(/C=C/c3ccc(O)c(O)c3Br)sc-2c1O]

In another embodiment, the compound is a compound represented by formula 21:

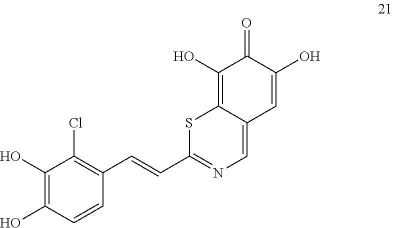 [SMILES: O=c1c(O)cc2cnc(/C=C/c3ccc(O)c(O)c3Cl)sc-2c1O]

In another embodiment, the compound is a compound represented by formula 22:

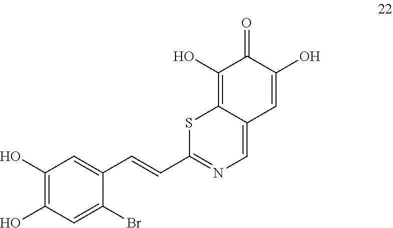 [SMILES: O=c1c(O)cc2cnc(/C=C/c3cc(O)c(O)cc3Br)sc-2c1O]

In another embodiment, the compound is a compound represented by formula 23:

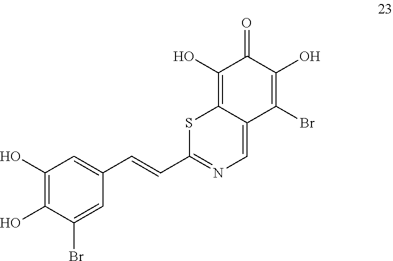 [SMILES: O=c1c(O)c2sc(/C=C/c3cc(O)c(O)c(Br)c3)ncc-2c(Br)c1O]

In another embodiment, the compound is a compound represented by formula 24:

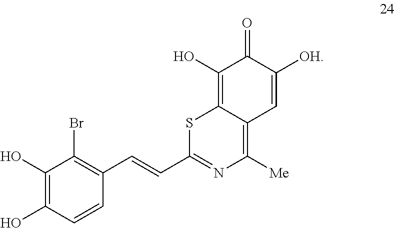 [SMILES: Cc1nc(/C=C/c2ccc(O)c(O)c2Br)sc2c(O)c(=O)c(O)cc1-2]

In another embodiment, the compound is a compound represented by formula 25:

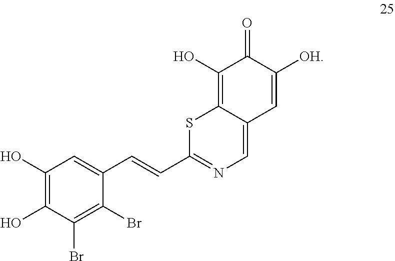 [SMILES: O=c1c(O)cc2cnc(/C=C/c3cc(O)c(O)c(Br)c3Br)sc-2c1O]

A currently preferred compound of formula (II) is a compound of formula 20.

In other embodiments, the compound is any of the derivatives described in A) PCT International Patent Application Publication No. WO 2008/068751; B) PCT International Patent Application Publication No. WO 2009/147682; or C) PCT International Patent Application No. WO 2012/090204. The contents of each of the aforementioned references are incorporated by reference herein in their entirety as if fully set forth herein.

It is understood that all conformers, geometrical isomers, stereoisomers, enantiomers and diastereomers of any of the compounds described herein, are encompassed and may be used in the combinations and methods described by the present application.

All stereoisomers of the above compounds are contemplated, either in admixture or in pure or substantially pure form. The compounds can have asymmetric centers at any of the atoms. Consequently, the compounds can exist in enantiomeric or diastereomeric forms or in mixtures thereof. The present invention contemplates the use of any racemates (i.e. mixtures containing equal amounts of each enantiomers), enantiomerically enriched mixtures (i.e., mixtures enriched for one enantiomer), pure enantiomers or diastereomers, or any mixtures thereof. The chiral centers can be designated as R or S or R,S or d,D, I,L or d,I, D,L. Compounds comprising amino acid residues include residues of D-amino acids, L-amino acids, or racemic derivatives of amino acids. Compounds comprising sugar residues include residues of D-sugars, L-sugars, or racemic derivatives of sugars. Residues of D-sugars, which appear in nature, are preferred. In addition, several of the compounds of the invention contain one or more double bonds. The present invention intends to encompass all structural and geometrical isomers including cis, trans, E and Z isomers, independently at each occurrence.

One or more of the compounds of the invention, may be present as a salt. The term “salt” encompasses both basic and acid addition salts, including but not limited to, carboxylate salts or salts with amine nitrogens, and include salts formed with the organic and inorganic anions and cations discussed below. Furthermore, the term includes salts that form by standard acid-base reactions with basic groups (such as amino groups) and organic or inorganic acids. Such acids include hydrochloric, hydrofluoric, trifluoroacetic, sulfuric, phosphoric, acetic, succinic, citric, lactic, maleic, fumaric, palmitic, cholic, pamoic, mucic, D-glutamic, D-camphoric, glutaric, phthalic, tartaric, lauric, stearic, salicylic, methanesulfonic, benzenesulfonic, sorbic, picric, benzoic, cinnamic, and like acids. Each possibility represents a separate embodiment of the invention.

The term “organic or inorganic cation” refers to counter-ions for the anion of a salt. The counter-ions include, but are not limited to, alkali and alkaline earth metals (such as lithium, sodium, potassium, barium, aluminum and calcium); ammonium and mono-, di- and tri-alkyl amines such as trimethylamine, cyclohexylamine; and the organic cations, such as dibenzylammonium, benzylammonium, 2-hydroxyethylammonium, bis(2-hydroxyethyl)ammonium, phenylethylbenzylammonium, dibenzylethylenediammonium, and like cations. See, for example, Berge et al., J. Pharm. Sci. (1977), 66:1-19, which is incorporated herein by reference.

The present invention also includes solvates of the compounds of the present invention and salts thereof. “Solvate” means a physical association of a compound of the invention with one or more solvent molecules. This physical association involves varying degrees of ionic and covalent bonding, including hydrogen bonding. In certain instances the solvate will be capable of isolation. “Solvate” encompasses both solution-phase and isolatable solvates. Non-limiting examples of suitable solvates include ethanolates, methanolates and the like. “Hydrate” is a solvate wherein the solvent molecule is water.

The present invention also includes polymorphs of the compounds of the present invention and salts thereof. The term “polymorph” refers to a particular crystalline or amorphous state of a substance, which can be characterized by particular physical properties such as X-ray diffraction, IR or Raman spectra, melting point, and the like.

Without being bound to any particular theory or mechanism of action, it is contemplated that the compounds of the present invention are inhibitors of PK signaling, such as IGF-1R. It has now been surprisingly found that these compounds, in addition to being inhibitors of IGF-1R, also lead to the dissociation of the IGF-1R substrates IRS1/2 from the cell membrane, inhibitory serine phosphorylation and/or degradation of the IRS1/2 proteins. This activity leads to long lasting inhibition of the IGF-1R and IR pathways, growth inhibition of a wide range of cancer cell types, and potent anti-tumor effects. These compounds are therefore referred to as “modulators of IRS”. In some embodiments, the compound of formula I is an inhibitor of an insulin receptor or an insulin-like growth factor-1 receptor (IGF-1R) signaling, and/or the compound of formula I interacts with, affects or inhibits a substrate protein in the IGF-1R mediated pathway. In some embodiments, the substrate protein is Insulin Receptor Substrate 1 (IRS1), Insulin Receptor Substrate 2 (IRS2), or a combination thereof. In one particular embodiment, the compound of formula I is an IGF-1R kinase inhibitor that leads to at least one of the dissociation of IRS1 or IRS2 from the cell membrane, phosphorylation of IRS1 or IRS2, and/or degradation of IRS1 or IRS2, in any order.

The compounds described herein are also modulators of signal transducer and activator of transcription 3 (Stat3). In some embodiments, the compounds lead to the inhibition of Stat3 phosphorylation in cancer cells. Increased levels of Stat3 phosphorylation are detected in various cancers and drug-resistant cancers, leading to enhanced cancer survival. Without wishing to be bound by any particular theory or mechanism of action it is contemplated that inhibiting Stat3 activity may synergize with such PK inhibitor drugs, which as a side effect upregulate Stat3, may prevent acquired resistance to such drugs and may be effective for drug-resistant cancers. Furthermore, Stat3 is often activated in cancer and directly involved in the implementation and maintenance of the cancer immunosuppressive microenvironment and plays a central role in tumor immune evasion. Without wishing to be bound by any particular theory or mechanism of action, it is contemplated that inhibition of Stat3 phosphorylation un-masks the tumor from the local immune system and sensitize them to.

### Chemical Definitions

An “alkyl” group refers to any saturated aliphatic hydrocarbon, including straight-chain and branched-chain alkyl groups. In one embodiment, the alkyl group has 1-4 carbons designated here as C₁-C₄-alkyl. The alkyl group may be unsubstituted or substituted by one or more groups selected from halogen, hydroxy, alkoxy carbonyl, amido, alkylamido, dialkylamido, nitro, amino, alkylamino, dialkylamino, carboxyl, thio and thioalkyl.

A “hydroxy” group refers to an OH group. An “alkoxy” group refers to an —O-alkyl group wherein R is alkyl as defined above.

An “amino” group refers to an NH 2 group. An alkylamino group refers to an —NHR group wherein R is alkyl is as defined above. A dialkylamino group refers to an —NRR′ group wherein R and R′ are alkyl as defined above.

An “amido” group refers to a —C(O)NH₂ group. An alkylamido group refers to an —C(O)NHR group wherein R is alkyl is as defined above. A dialkylamido group refers to an —C(O)NRR′ group wherein R and R′ are alkyl as defined above.

The term “halogen” or “halo” as used herein alone or as part of another group refers to chlorine, bromine, fluorine, and iodine. The term “haloalkyl” refers to an alkyl group having some or all of the hydrogens independently replaced by a halogen group including, but not limited to, trichloromethyl, tribromomethyl, trifluoromethyl, triiodomethyl, difluoromethyl, chlorodifluoromethyl, pentafluoroethyl, 1,1-difluoroethyl bromomethyl, chloromethyl, fluoromethyl, iodomethyl, and the like.

Within the scope of the present invention are prodrugs of the compounds disclosed herein. The term “prodrug” represents compounds which are rapidly transformed in vivo to any of compounds represented by formula I, for example by hydrolysis in the blood. Thus, the term “prodrug” refers to a precursor of any of the compounds of the present invention that is pharmaceutically acceptable. A prodrug may be inactive when administered to a subject, but is converted in vivo to an active compound. The use of prodrugs is particularly advantageous for facilitating the administration of the compounds. The prodrug compound often offers benefits of solubility, tissue compatibility or delayed release in a mammalian organism.

### Treatment of Cancer

In one embodiment, the present invention relates to method of sensitizing a tumor to immunotherapy by an anti-programmed cell death protein 1 (PD-1) antibody, an anti-programmed cell death protein 1 ligand (PD-L1) antibody or a combination thereof, the method comprising the step of contacting the tumor with a compound represented by the structure of formula (I), or salts or hydrates thereof, in combination with an anti-programmed cell death protein 1 (PD-1) antibody and/or anti-programmed cell death protein 1 ligand (PD-L1) antibody.

In another embodiment, the present invention relates to method of sensitizing a tumor to immunotherapy by an anti-programmed cell death protein 1 (PD-1) antibody, an anti-programmed cell death protein 1 ligand (PD-L1) antibody or a combination thereof, the method comprising the step of contacting the tumor with a compound represented by the structure of formula (II), or salts or hydrates thereof, in combination with an anti-programmed cell death protein 1 (PD-1) antibody and/or anti-programmed cell death protein 1 ligand (PD-L1) antibody.

In other embodiments, the present invention further relates to a combination comprising a compound of formula (I) with an anti-programmed cell death protein 1 (PD-1) antibody, an anti-programmed cell death protein 1 ligand (PD-L1) antibody or a combination thereof, for use in sensitizing a tumor to immunotherapy with the anti-PD-1 and/or anti-PD-L1 antibody.

In other embodiments, the present invention further relates to a combination comprising a compound of formula (II) with an anti-programmed cell death protein 1 (PD-1) antibody, an anti-programmed cell death protein 1 ligand (PD-L1) antibody or a combination thereof, for use in sensitizing a tumor to immunotherapy with the anti-PD-1 and/or anti-PD-L1 antibody.

A currently preferred compound for use in the combinations of the present invention is a compound of formula 4. Thus, in another embodiment, the present invention further relates to method of sensitizing a tumor to immunotherapy by an anti-programmed cell death protein 1 (PD-1) antibody, an anti-programmed cell death protein 1 ligand (PD-L1) antibody or a combination thereof, the method comprising the step of contacting the tumor with a compound represented by the structure of formula 4, or salts or hydrates thereof, in combination with an anti-PD-1 and/or anti-PD-L1 antibody.

In other embodiments, the present invention further relates to a combination comprising a compound of formula 4 with an anti-programmed cell death protein 1 (PD-1) antibody, an anti-programmed cell death protein 1 ligand (PD-L1) antibody or a combination thereof, for use in sensitizing a tumor to immunotherapy with the anti-PD-1 and/or anti-PD-L1 antibody.

Another currently preferred compound for use in the combinations of the present invention is a compound of formula 20. Thus, in another embodiment, the present invention further relates to method of sensitizing a tumor to immunotherapy by an anti-programmed cell death protein 1 (PD-1) antibody, an anti-programmed cell death protein 1 ligand (PD-L1) antibody or a combination thereof, the method comprising the step of contacting the tumor with a compound represented by the structure of formula 20, or salts or hydrates thereof, in combination with the anti-PD-1 and/or anti-PD-L1 antibody.

In other embodiments, the present invention further relates to a combination comprising a compound of formula 20 with an anti-programmed cell death protein 1 (PD-1) antibody, an anti-programmed cell death protein 1 ligand (PD-L1) antibody or a combination thereof, for use in sensitizing a tumor to immunotherapy with the anti-PD-1 and/or anti-PD-L1 antibody.

In some embodiments, the tumor is present in a cancer patient who is receiving immunotherapy or is a candidate for receiving immunotherapy.

Currently preferred combinations are combinations of Pembrolizumab (Keytruda®) and a compound of formula 4.

Other currently preferred combinations are combinations of Pembrolizumab (Keytruda®) and a compound of formula 20.

The term “cancer” as used herein refers to a disorder in which a population of cells has become, in varying degrees, unresponsive to the control mechanisms that normally govern proliferation and differentiation. Cancer refers to various types of malignant neoplasms and tumors, including primary tumors, and tumor metastasis. Non-limiting examples of cancers which can be treated by the combinations of the present invention are brain, ovarian, colorectal, pancreatic, head and neck, esophagus, prostate, kidney, bladder, breast, lung, oral, and skin cancers.

The combinations of the present invention are suitable for treating various types of cancers. In particular, the combinations of the present invention are active against head and neck (H&N) cancer, sarcoma, multiple myeloma, ovarian cancer, breast cancer, kidney cancer, stomach cancer, hematopoietic cancers, lymphoma, leukemia, including lymphoblastic leukemia, lung carcinoma, melanoma, glioblastoma, hepatocarcinoma, pancreatic cancer, easopageal cancer, prostate cancer and colon cancer. Each possibility represents a separate embodiment of the present invention.

Other examples of cancers are: carcinomas, sarcomas, myelomas, leukemias, lymphomas and mixed type tumors. Particular categories of tumors include lymphoproliferative disorders, breast cancer, ovarian cancer, prostate cancer, cervical cancer, endometrial cancer, bone cancer, liver cancer, stomach cancer, colon cancer, pancreatic cancer, cancer of the thyroid, head and neck cancer, cancer of the central nervous system, cancer of the peripheral nervous system, skin cancer, kidney cancer, as well as metastases of all the above. Particular types of tumors include hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, squamous cell carcinoma, basal cell carcinoma, adenocarcinoma (well differentiated, moderately differentiated, poorly differentiated or undifferentiated), renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma, and hepatocarcinoma. Each possibility represents a separate embodiment of the present invention.

The term “treatment of cancer” in the context of the present invention includes at least one of the following: a decrease in the rate of growth of the cancer (i.e. the cancer still grows but at a slower rate); cessation of growth of the cancerous growth, i.e., stasis of the tumor growth, and, in preferred cases, the tumor diminishes or is reduced in size. The term also includes reduction in the number of metastases, reduction in the number of new metastases formed, slowing of the progression of cancer from one stage to the other and a decrease in the angiogenesis induced by the cancer. In most preferred cases, the tumor is totally eliminated. Additionally included in this term is lengthening of the survival period of the subject undergoing treatment, lengthening the time of diseases progression, tumor regression, and the like. It is to be understood that the term “treating cancer” also refers to the inhibition of a malignant (cancer) cell proliferation including tumor formation, primary tumors, tumor progression or tumor metastasis. The term “inhibition of proliferation” in relation to cancer cells, may further refer to a decrease in at least one of the following: number of cells (due to cell death which may be necrotic, apoptotic or any other type of cell death or combinations thereof) as compared to control; decrease in growth rates of cells, i.e. the total number of cells may increase but at a lower level or at a lower rate than the increase in control; decrease in the invasiveness of cells (as determined for example by soft agar assay) as compared to control even if their total number has not changed; progression from a less differentiated cell type to a more differentiated cell type; a deceleration in the neoplastic transformation; or alternatively the slowing of the progression of the cancer cells from one stage to the next.

As used herein, the term “administering” refers to bringing in contact with the combination of the present invention. Administration can be accomplished to cells or tissue cultures, or to living organisms, for example humans. In one embodiment, the present invention encompasses administering the combinations of the present invention to a human subject.

A “therapeutic” treatment is a treatment administered to a subject who exhibits signs of pathology for the purpose of diminishing or eliminating those signs. A “therapeutically effective amount” is that amount of compound or a composition which is sufficient to provide a beneficial effect to the subject to which the compound or composition is administered.

The term “following cease of treatment” as used herein means after treatment with the drug of choice is stopped. For example, according to certain embodiments of the present invention, the IRS/Stat3 Dual Modulator (e.g., compound of formula (I) or (II)) is administered together (sequentially or concurrently) with any of the combination treatments described herein, for a desired duration of time. Then, treatment (with all compounds) is stopped and the tumors are monitored for a desired period of time. As contemplated herein, the IRS/Stat3 Dual Modulators of the present invention are able to prevent or delay tumor recurrence following cease of treatment with the any of the combination drugs described herein, to a greater extent than any of these drugs administered alone.

The term “treating a tumor that has developed resistance” to a certain anti-cancer drug, or “preventing acquired resistance of a tumor” to a certain anti-cancer drug, means any one or more of the following: (i) the tumors acquire or develop resistance as a result of treatment to that anti-cancer drug; (ii) that the tumors acquire or develop resistance as a result of treatment with other anti-cancer drugs; or (iii) the tumors have a primary resistance to that anti-cancer drug.

The combination therapy can provide a therapeutic advantage in view of the differential toxicity associated with the two individual treatments. For example, treatment with one compound can lead to a particular toxicity that is not seen with the other compound, and vice versa. As such, this differential toxicity can permit each treatment to be administered at a dose at which the toxicities do not exist or are minimal, such that together the combination therapy provides a therapeutic dose while avoiding the toxicities of each of the constituents of the combination agents. Furthermore, when the therapeutic effects achieved as a result of the combination treatment are enhanced or synergistic, i.e., significantly better than additive therapeutic effects, the doses of each of the agents can be reduced even further, thus lowering the associated toxicities to an even greater extent.

The terms “synergistic”, “cooperative” and “super-additive” and their various grammatical variations are used interchangeably herein. An interaction between an IRS/Stat3 dual modulator and the anti-PD-1 and/or anti-PD-L1 antibody is considered to be synergistic, cooperative or super-additive when the observed effect (e.g., cytotoxicity) in the presence of the drugs together is higher than the sum of the individual effects of each drug administered separately. In one embodiment, the observed combined effect of the drugs is significantly higher than the sum of the individual effects. The term significant means that the observed p<0.05. A non-limiting manner of calculating the effectiveness of the combined treatment comprises the use of the Bliss additivism model (Cardone et al. Science (1998), 282: 1318-1321) using the following formula: Ebliss=EA+EB−EA×EB, where EA and EB are the fractional inhibitions obtained by drug A alone and drug B alone at specific concentrations. When the experimentally measured fractional inhibition is equal to Ebliss, the combination provides an additive therapeutic effect. When the experimentally measured fractional inhibition is greater than Ebliss, the combination provides a synergistic therapeutic effect.

### Pharmaceutical Compositions

Although the components of the combinations of the present invention can be administered alone, it is contemplated that the components are administered in pharmaceutical compositions further containing at least one pharmaceutically acceptable carrier or excipient. Each of the components can be administered in a separate pharmaceutical composition, or the combination can be administered in one pharmaceutical composition.

The pharmaceutical compositions of the present invention can be formulated for administration by a variety of routes including oral, rectal, transdermal, parenteral (subcutaneous, intraperitoneal, intravenous, intra-arterial, transdermal and intramuscular), topical, intranasal, or via a suppository. Each possibility represents a separate embodiment of the present invention. Such compositions are prepared in a manner well known in the pharmaceutical art and comprise as an active ingredient at least one compound of the present invention as described hereinabove, and a pharmaceutically acceptable excipient or a carrier. The term “pharmaceutically acceptable” means approved by a regulatory agency of the Federal or a state government or listed in the U.S. Pharmacopeia or other generally recognized pharmacopeia for use in animals and, more particularly, in humans.

During the preparation of the pharmaceutical compositions according to the present invention the active ingredient is usually mixed with a carrier or excipient, which may be a solid, semi-solid, or liquid material. The compositions can be in the form of tablets, pills, capsules, pellets, granules, powders, lozenges, sachets, cachets, elixirs, suspensions, dispersions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. Each possibility represents a separate embodiment of the present invention.

The carriers may be any of those conventionally used and are limited only by chemical-physical considerations, such as solubility and lack of reactivity with the compound of the invention, and by the route of administration. The choice of carrier will be determined by the particular method used to administer the pharmaceutical composition. Some examples of suitable carriers include lactose, glucose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water and methylcellulose. Each possibility represents a separate embodiment of the present invention. The formulations can additionally include lubricating agents such as talc, magnesium stearate, and mineral oil; wetting agents, surfactants, emulsifying and suspending agents; preserving agents such as methyl- and propylhydroxybenzoates; sweetening agents; flavoring agents, colorants, buffering agents (e.g., acetates, citrates or phosphates), disintegrating agents, moistening agents, anti-bacterial agents, anti-oxidants (e.g., ascorbic acid or sodium bisulfite), chelating agents (e.g., ethylenediaminetetraacetic acid), and agents for the adjustment of tonicity such as sodium chloride. Other pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions. Each possibility represents a separate embodiment of the present invention.

For preparing solid compositions such as tablets, the principal active ingredient(s) is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing, for example, from about 0.1 mg to about 2000 mg, from about 0.1 mg to about 500 mg, from about 1 mg to about 100 mg, from about 100 mg to about 250 mg, etc. of the active ingredient(s) of the present invention.

Any method can be used to prepare the pharmaceutical compositions. Solid dosage forms can be prepared by wet granulation, dry granulation, direct compression and the like. The solid dosage forms of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate. Each possibility represents a separate embodiment of the present invention.

The liquid forms in which the compositions of the present invention may be incorporated, for administration orally or by injection, include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles. Each possibility represents a separate embodiment of the present invention.

Compositions for inhalation or insulation include solutions and suspensions in pharmaceutically acceptable aqueous or organic solvents, or mixtures thereof, and powders. The liquid or solid compositions may contain suitable pharmaceutically acceptable excipients as described above. In one embodiment, the compositions are administered by the oral or nasal respiratory route for local or systemic effect. Compositions in pharmaceutically acceptable solvents may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered, orally or nasally, from devices that deliver the formulation in an appropriate manner.

Another formulation suitable for the compositions and methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art.

In yet another embodiment, the composition is prepared for topical administration, e.g. as an ointment, a gel a drop or a cream. For topical administration to body surfaces using, for example, creams, gels, drops, ointments and the like, the compounds of the present invention can be prepared and applied in a physiologically acceptable diluent with or without a pharmaceutical carrier. The present invention may be used topically or transdermally to treat cancer, for example, melanoma. Adjuvants for topical or gel base forms may include, for example, sodium carboxymethylcellulose, polyacrylates, polyoxyethylene-polyoxypropylene-block polymers, polyethylene glycol and wood wax alcohols. Each possibility represents a separate embodiment of the present invention.

Alternative formulations include nasal sprays, liposomal formulations, slow-release formulations, pumps delivering the drugs into the body (including mechanical or osmotic pumps) controlled-release formulations and the like, as are known in the art.

The compositions are preferably formulated in a unit dosage form. The term “unit dosage forms” refers to physically discrete units suitable as unitary dosages for human subjects and other mammals, each unit containing a predetermined quantity of active material(s) calculated to produce the desired therapeutic effect, in association with a suitable pharmaceutical excipient.

In preparing a formulation, it may be necessary to mill the active ingredient to provide the appropriate particle size prior to combining with the other ingredients. If the active compound is substantially insoluble, it ordinarily is milled to a particle size of less than 200 mesh. If the active ingredient is substantially water soluble, the particle size is normally adjusted by milling to provide a substantially uniform distribution in the formulation, e.g. about 40 mesh.

It may be desirable to administer the pharmaceutical composition of the invention locally to the area in need of treatment; this may be achieved by, for example, and not by way of limitation, local infusion during surgery, infusion to the liver via feeding blood vessels with or without surgery, topical application, e.g., in conjunction with a wound dressing after surgery, by injection, by means of a catheter, by means of a suppository, or by means of an implant, the implant being of a porous, non-porous, or gelatinous material. According to some embodiments, administration can be by direct injection e.g., via a syringe, at the site of a tumor or neoplastic or pre-neoplastic tissue.

The compounds may also be administered by any convenient route, for example by infusion or bolus injection, by absorption through epithelial linings (e.g., oral mucosa, rectal and intestinal mucosa, etc.), and may be administered together with other therapeutically active agents. The administration may be localized or it may be systemic. In addition, it may be desirable to introduce the pharmaceutical compositions of the invention into the central nervous system by any suitable route, including intra-ventricular and intrathecal injection; intra-ventricular injection may be facilitated by an intra-ventricular catheter, for example, attached to a reservoir. Pulmonary administration can also be employed, e.g., by use of an inhaler or nebulizer, and formulation with an aerosolizing agent.

A compound of the present invention can be delivered in an immediate release or in a controlled release system. In one embodiment, an infusion pump may be used to administer a compound of the invention, such as one that is used for delivering chemotherapy to specific organs or tumors (see Buchwald et al., 1980, Surgery 88: 507; Saudek et al., 1989, N. Engl. J. Med. 321: 574). In one embodiment, a compound of the invention is administered in combination with a biodegradable, biocompatible polymeric implant, which releases the compound over a controlled period of time at a selected site. Examples of polymeric materials include, but are not limited to, polyanhydrides, polyorthoesters, polyglycolic acid, polylactic acid, polyethylene vinyl acetate, copolymers and blends thereof. In yet another embodiment, a controlled release system can be placed in proximity of the therapeutic target, thus requiring only a fraction of the systemic dose.

Furthermore, at times, the pharmaceutical compositions may be formulated for parenteral administration (subcutaneous, intravenous, intra-arterial, transdermal, intraperitoneal or intramuscular injection) and may include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. Oils such as petroleum, animal, vegetable, or synthetic oils and soaps such as fatty alkali metal, ammonium, and triethanolamine salts, and suitable detergents may also be used for parenteral administration. The above formulations may also be used for direct intra-tumoral injection. Further, in order to minimize or eliminate irritation at the site of injection, the compositions may contain one or more nonionic surfactants. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described and known in the art. Each possibility represents a separate embodiment of the present invention.

Alternatively, the combinations of the present invention can be used in hemodialysis such as leukophoresis and other related methods, e.g., blood is drawn from the patient by a variety of methods such as dialysis through a column/hollow fiber membrane, cartridge etc., is treated with the IRS/Stat3 dual modulator and/or additional anti-cancer agent ex-vivo, and returned to the patient following treatment. Such treatment methods are well known and described in the art. See, e.g., Kolho et al. (J. Med. Virol. 1993, 40(4):318-21); Ting et al. (Transplantation, 1978, 25(1):31-3); the contents of which are hereby incorporated by reference in their entirety.

### Doses and Dosing Schedules

The treatment with the IRS/Stat3 dual modulator and the anti-PD-1 and/or anti-PD-L1 antibody can take place sequentially in any order, simultaneously or a combination thereof. For example, administration of an IRS/Stat3 dual modulator can take place prior to, after or at the same time as administration of the anti-PD-1 and/or anti-PD-L1 antibody. For example, a total treatment period can be decided for the IRS/Stat3 dual modulator. The anti-PD-1 and/or anti-PD-L1 antibody can be administered prior to onset of treatment with the IRS/Stat3 dual modulator or following treatment with the IRS/Stat3 dual modulator. In addition, the anti-PD-1 and/or anti-PD-L1 antibody can be administered during the period of IRS/Stat3 dual modulator administration but does not need to occur over the entire treatment period. In another embodiment, the treatment regimen includes pre-treatment with the anti-PD-1 and/or anti-PD-L1 antibody or the IRS/Stat3 dual modulator followed by the addition of the other agent or agents. Alternating sequences of administration are also contemplated. Alternating administration includes administration of an IRS/Stat3 dual modulator and other anti-PD-1 and/or anti-PD-L1 antibody in alternating sequences, e.g., IRS/Stat3 dual modulator, followed by the anti-PD-1 and/or anti-PD-L1 antibody t, followed by IRS/Stat3 dual modulator, etc.

The amount of a compound that will be effective in the treatment of a particular disorder or condition, including cancer, will depend on the nature of the disorder or condition, and can be determined by standard clinical techniques. In addition, in vitro assays may optionally be employed to help identify optimal dosage ranges. The precise dose to be employed in the formulation will also depend on the route of administration, and the progression of the disease or disorder, and should be decided according to the judgment of the practitioner and each patient's circumstances. A preferred dosage will be within the range of 0.01-1000 mg/kg of body weight, 0.1mg/kg to 100 mg/kg, 1 mg/kg to 100mg/kg, 10 mg/kg to 75 mg/kg, 0.1-1 mg/kg, etc. (non-limiting) amounts of the IRS/Stat3 dual modulator and anti-PD-1 and/or anti-PD-L1 antibody include 0.1 mg/kg, 0.2 mg/kg, 0.5 mg/kg, 1 mg/kg, 5 mg/kg, 10 mg/kg, 20 mg/kg, 50 mg/kg, 60 mg/kg, 75 mg/kg and 100 mg/kg. Alternatively, the amount administered can be measured and expressed as molarity of the administered compound. By way of illustration and not limitation, an IRS/Stat3 dual modulator (e.g. a compound of any of formulae I, II, III, IV) can be administered in a range of 0.1-10 mM, e.g., 0.1, 0.25, 0.5, 1 and 2 mM. Alternatively, the amount administered can be measured and expressed as mg/ml, or ng/ml. By way of illustration and not limitation, the anti-PD-1 and/or anti-PD-L1 antibody can be administered in an amount of 1 ng/ml to 100 mg/ml, for example 1-1000 ng/ml, 1-100 ng/ml, 1-1000 [symbol] g/ml, 1-100 [symbol] g/ml, 1-1000 mg/ml, 1-100 mg/ml, etc. Effective doses may be extrapolated from dose-response curves derived from in vitro or animal model test bioassays or systems. When a synergistic effect is observed, the overall dose of each of the components may be lower, thus the side effects experienced by the subject may be significantly lower, while a sufficient anti-cancer effect is nevertheless achieved.

In one embodiment, the combination therapy reduces the amount of each of its component by a factor of 2, i.e., each component is given at half the dose as compared with single agent therapy, and still achieves the same or similar therapeutic effect. In another embodiment, the combination therapy reduces the amount of each of its component by a factor of 5, 10, 20, 50 or 100.

The administration schedule will depend on several factors such as the cancer being treated, the severity and progression, the patient population, age, weight etc. For example, the compositions of the invention can be taken once-daily, twice-daily, thrice daily, once-weekly or once-monthly. In addition, the administration can be continuous, i.e., every day, or intermittently. The terms “intermittent” or “intermittently” as used herein means stopping and starting at either regular or irregular intervals. For example, intermittent administration can be administration one to six days per week or it may mean administration in cycles (e.g. daily administration for two to eight consecutive weeks, then a rest period with no administration for up to one week) or it may mean administration on alternate days. The different components of the combination can, independently of the other, follow different dosing schedules.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXPERIMENTAL EXAMPLES

### Example 1: Combination of Compound 4, in Combination With Pembolizumab (Keytruda®), Converts Non-Responding Tumors to Responders and Block Tumor Progression

### Experimental System:

Patient-derived xenograft (PDX) of esophagogastric junction adenocarcinoma biopsy subcutaneous (SC) implanted into NodScid mice. At the first day of treatment peripheral blood mononuclear cells (PBMCs) from the same patient were intravenous injected to the mice (double autologous).

Fresh human primary esophagogastric junction adenocarcinoma biopsy was subcutaneously (SC) implanted into NSG mice (P0).

Subcutaneous implantation of human primary tumor biopsy into NodScid mice.

The tumor (P3) was extracted from the mice, measured, cut into small pieces of 1-2 mm and transferred into the gentleMACS Tube containing the sterile saline. Tumor volume was adjusted with saline to 1.5 mm³ tumor volume/100 ul saline. The sample was dissociated using the gentle MACS Octo Dissociator. The dissociated tumor tissue was collected with 18G syringe and injected (approximately 1.5 mm³ tumor volume/100 ul saline per mouse) under the skin to male NOD.CB17-Prkdcscid/J (NodScid) mice. Animals were observed and monitored for any discomfort and immobility day by day.

Isolation of human PBMCs: Human PBMCs were isolated from heparinized venous blood samples of the same esophagus adenocarcinoma patient by density gradient centrifugation method using Ficol Histopaque (Sigma). Briefly, the heparinized blood was layered on LSM medium gently in the ratio of 2:1 (e.g. 10 ml blood on 10 ml ficol) and subjected to centrifugation at 800 g for 10 minutes at RT (centrifuge' setting: swing-rotor, no brake). The white layer representing PBMCs was aspirated out gently and transferred aseptically into sterile centrifuge tubes. The suspension of cells was then washed twice in order to dilute possible Ficol residues by adding 10 ml PBS and centrifugation at 600 g for 10 minutes. The cell pellet was resuspended in 0.5 ml PBS and subjected to cell counting, using trypan blue and hemocytometer. The total cell number and the number of viable cells were evaluated. The cells were separated for cryopreservation in filtered sterile 10% DMSO/FBS solution, placed in pre-cooled 4° C. Mr. Frosty in −80° C. overnight and then transferred to −80° C. On the day of injection, the cells were thawed, washed twice in order to dilute possible DMSO residues by the addition of PBS and centrifugation (600 g for 10 minutes, RT) and viable cells were counted.

Over 90% of the injected mice developed tumors and treatments initiated 7 days later when tumors reached average size of about 160 mm³ (day 0).

Treatments: PBMCs (2.1M viable cells/mouse) were injected to 12 mice on day 0 and 2 hr later the following treatments:
1. Control (20% HPbCD) 50 μl IV on days 0, 5 and 11 (3 mice)
2. Compound 4 70 mg/kg IV on days 0, 5 and 11 (3 mice)
3. Keytruda 6 mg/kg IP on days 0, 5 and 11 (3 mice)
4. Keytruda 6 mg/kg IP+Compound 4 70 mg/kg IV on days 0, 5 and 11 (3 mice)
*Keytruda was administrated ˜4 h following compound 4

All treatments for each of the treatment groups 1-4 were initiated simultaneously.

The length (l) and the width (w) of the tumors were measured 4-5 times a week and the volumes of the tumors were calculated as follows: v=lw²/2. Graphs represent mean tumor volumes with standard errors. Mice were examined 5 times a week and weighted at least twice a week.

Mice were sacrificed on day 18 and tumors were taken for analysis.

A parallel experiment of esophagogastric junction adenocarcinoma PDX without PBMCs of the patient, was conducted and the progression rate of the tumors in the control group was similar to the progression rate of the control group in the presence of PBMCs.

### Results

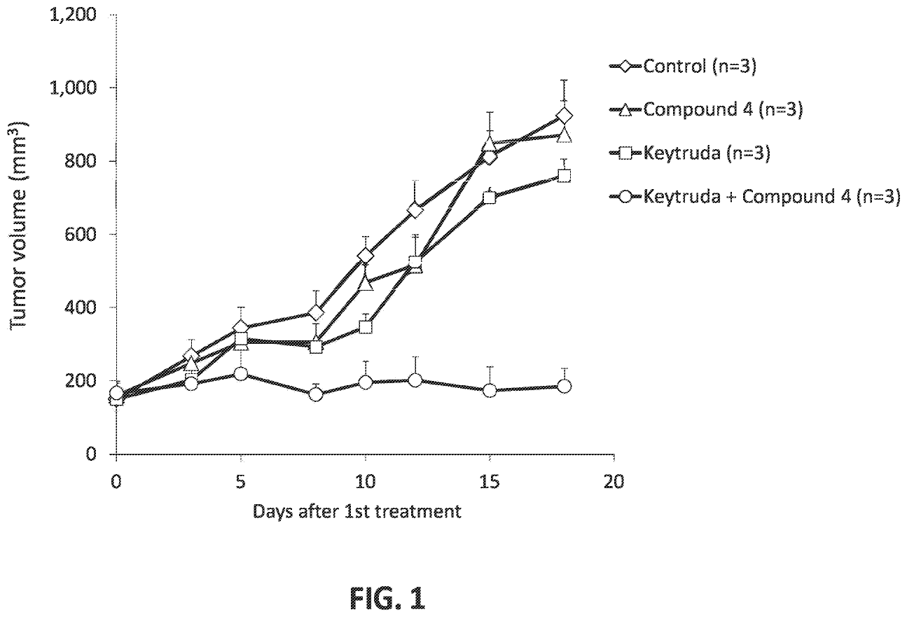

The study was conducted in a patient-derived xenograft (PDX) model of immune-deficient mice, in which a tumor originated from an esophagus cancer biopsy was implanted, and the mice were supplemented with immune cells from the same patient (double autologous). As shown in FIG. 1, while no response was observed with Keytruda® alone or with compound 4 alone, and the tumors aggressively progressed, mice treated with a combination of Keytruda® and compound 4 demonstrated complete blockage of tumor progression (Tumor growth inhibition=98%, p value=0.002). Thus, compound 4 in combination with Keytruda®, converted non-responding tumors to responders and blocked tumor progression in an immune-oncology preclinical model.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

What is claimed:

1. A pharmaceutical combination comprising a compound represented by the structure of formula (4): 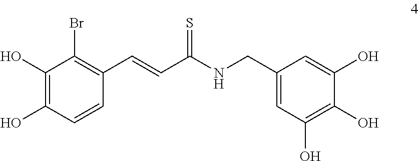 [SMILES: Oc1cc(CNC(=S)/C=C/c2ccc(O)c(O)c2Br)cc(O)c1O]
or salts or hydrates thereof, in combination with an anti-programmed cell death protein 1 (PD-1) antibody, an anti-programmed cell death protein 1 ligand (PD-L1) antibody or a combination thereof.

2. The combination according to claim 1, wherein the anti-PD-1 antibody is selected from the group consisting of Pembrolizumab (Keytruda), Nivolumab (Opdivo), AGEN-2034, AMP-224, BCD-100, BGBA-317, BI-754091, CBT-501, CC-90006, Cemiplimab, GLS-010, IBI-308, JNJ-3283, JS-001, MEDI-0680, MGA-012, MGD-013, PDR-001, PF-06801591, REGN-2810, SHR-1210, TSR-042, LZM-009, ABBV-181, and Pidilizumab.

3. The combination according to claim 2, wherein the anti-PD-1 antibody is Pembrolizumab (Keytruda).

4. The combination according to claim 2, wherein the anti-PD-1 antibody is Nivolumab (Opdivo).

5. The combination according to claim 1, wherein the anti-PD-L1 antibody is selected from the group consisting of Avelumab (Bavencio), Durvalumab (Imfinzi), Atezolizumab (Tecentriq), BMS-936559, CX-072, SHR-1316, M-7824, LY-3300054, FAZ-053, KN-035, CA-170, CK-301, CS-1001, HLX-10, MCLA-145, MSB-2311 and MEDI-4736.

6. A method of sensitizing a tumor to immunotherapy by an anti-programmed cell death protein 1 (PD-1) antibody, anti-programmed cell death protein 1 ligand (PD-L1) antibody, or a combination thereof, the method comprising the step of contacting the tumor with the combination according to claim 1.

7. The method according to claim 6, wherein the anti-PD-1 antibody is selected from the group consisting of Pembrolizumab (Keytruda), Nivolumab (Opdivo), AGEN-2034, AMP-224, BCD-100, BGBA-317, BI-754091, CBT-501, CC-90006, Cemiplimab, GLS-010,1131-308, JNJ-3283, JS-001, MEDI-0680, MGA-012, MGD-013, PDR-001, PF-06801591, REGN-2810, SHR-1210, TSR-042, LZM-009, ABBV-181, and Pidilizumab.

8. The method according to claim 7, wherein the anti-PD-1 antibody is Pembrolizumab (Keytruda).

9. The method according to claim 7, wherein the anti-PD-1 antibody is Nivolumab (Opdivo).

10. The method according to claim 6, wherein the anti-PD-L1 antibody is selected from the group consisting of Avelumab (Bavencio), Durvalumab (Imfinzi), Atezolizumab (Tecentriq), BMS-936559, CX-072, SHR-1316, M-7824, LY-3300054, FAZ-053, KN-035, CA-170, CK-301, CS-1001, HLX-10, MCLA-145, MSB-2311 and MEDI-4736.

11. The method according to claim 6, wherein the tumor is resistant to treatment with the anti-PD-1 and/or anti-PD-L1 antibody alone.

12. The method according to claim 6, wherein the compound re-sensitizes the tumor to immunotherapy by the anti-PD-1 and/or anti-PD-L1 antibody, by enhancing response of the tumor to the anti-PD-1 and/or anti-PD-L1 antibody, converting non-responding tumors to responders and/or blocking tumor progression.

13. The method according to claim 6, wherein the tumor is present in a cancer patient who is receiving anti-PD-1 and/or anti-PD-L1 immunotherapy or is a candidate for receiving such immunotherapy.

14. The method according to claim 13, wherein the cancer is selected from the group consisting of head and neck (H&N) cancer, esophagus cancer, sarcoma, multiple myeloma, ovarian cancer, breast cancer, kidney cancer, stomach cancer, hematopoietic cancers, lymphoma, leukemia, including lymphoblastic leukemia, lung carcinoma, melanoma, glioblastoma, hepatocarcinoma, prostate cancer, pancreatic cancer and colon cancer.

15. The method according to claim 6, wherein the compound of formula (4) and the anti-PD-1 and/or anti-PD-L1 antibody are administered in the same pharmaceutical composition.

16. The method according to claim 6, wherein the compound of formula (4) and the anti-PD-1 and/or anti-PD-L1 antibody are administered in separate pharmaceutical compositions, simultaneously or sequentially, in any order.
